(19) United States
(12) Patent Application Publication
(10) Pub. No.: US 2009/0209986 A1
(43) Pub. Date: Aug. 20, 2009

(54) Devices, Tools and Methods for Atrial Appendage Exclusion

(76) Inventors: Michael C. Stewart, San Jose, CA (US); Mark Juravic, San Francisco, CA (US); Albert K. Chin, Palo Alto, CA (US)

Correspondence Address: LAW OFFICE OF ALAN W. CANNON, 942 MESA OAK COURT, SUNNYVALE, CA, 94086, US

(21) Appl. No.: 12/372,707

(22) Filed: Feb. 17, 2009

Related U.S. Application Data
(60) Provisional application No. 61/028,952, filed on Feb. 15, 2008.

(51) Int. Cl. A61B 17/08

(52) U.S. Cl. 606/157

(57) ABSTRACT

Devices, tools and methods for occluding fluid flow between two walls of tissue in a patient. Two walls of tissue are compressed together with sufficient compressive force to prevent fluid flow between the two walls, while ensuring that the compressive force is not so great as to cause tissue necrosis. The devices, tools and methods may be carried out using minimally invasive surgical techniques, such as in reduced-access surgical sites. Devices, tools and methods are provided for occluding an atrial appendix.

### CROSS-REFERENCE

This application claims the benefit of U.S. Provisional Application No. 61/028,952, filed Feb. 15, 2008, Which application is incorporated herein, in its entirety, by reference thereto.

### FIELD OF THE INVENTION

The field of the present invention is apparatus and methods for performing minimally invasive surgery, more particularly to cardiac procedures performed with minimally invasive surgical techniques and apparatus.

### BACKGROUND OF THE INVENTION

More than two million Americans suffer from a type of cardiac arrhythmia called atrial fibrillation (“AF”). In AF, abnormal electrical impulses, in the atria, can cause the ventricles to contract rapidly and erratically, potentially compromising blood flow and sometimes causing fainting orthostatic hypotension (low blood pressure on standing up) or low blood pressure. Because the atria stop beating effectively during AF, they no longer empty completely with each beat. The remaining blood frequently pools in the atria and eventually clots. If a piece of an atrial blood clot breaks off, enters the circulation, and becomes lodged in an artery in the brain, a stroke results, often with extremely serious consequences.

AF significantly increases the risk of stroke, and about fifteen percent of all strokes occur in people with AF. Patients with AF are five times more likely to suffer a stroke than patients with normal atrial rhythm. Patients suffering from both AF and mitral stenosis (i.e., narrowing or blockage of the opening of the mitral valve connecting the left atrium and left ventricle) are seventeen times more likely to suffer a stroke. AF can be treated in several different ways, including by medication (e.g., beta-blockers, calcium antagonists, and the like), electrical cardioversion, implantation of an atrial pacemaker, or radiofrequency ablation to destroy the cardiac foci triggering the aberrant electrical signals.

Physicians commonly treat the increased risk of stroke in patients with AF with anticoagulant or antiplatelet medications to thin the blood and make it less prone to clotting. Long-term use of anticoagulants/antiplatelets (e.g. warfarin or aspirin) in patients with AF and other stroke risk factors can significantly reduce the incidence of strokes, although such drugs often have serious side effects. Interestingly, ninety percent of blood clots (“thrombi”) found in patients suffering from chronic AF originate from the Left Atrial Appendage (“LAA”), an endocrine organ located between the pulmonary artery and the left ventricle that regulates the relationship between pressure and volume in the left atrium, and plays a role in regulating cardiac output. Because most thrombi in patients with chronic AF originate in the LAA, occlusion of the LAA could greatly reduce the risk of stroke in AF patients, while eliminating the need for long-term use of anticoagulants.

Conventional methods for occluding the LAA require extremely invasive surgical procedures (e.g., opening the chest cavity deflation of a lung, and the like). Accordingly, a system for delivering an LAA occluding device by a minimally-invasive procedure would be beneficial

In patients undergoing therapy for atrial fibrillation, for example atrial ablation therapy, it is desirable to exclude the left atrial appendage from the circulatory path, such as by sealing off the appendage from the remainder of the atrial cavity, or removing the appendage from the atrium. Even when atrial ablation is performed in an attempt to cure atrial ablation, atrial appendage exclusion is still generally performed. In the event that the atrial ablation procedure is unsuccessful, the potential of stroke and other complications mentioned above is reduced in the patient with continuing atrial fibrillation that has bad the left atrial appendage excluded.

One current technique for excluding the left atrial appendage is by suturing along the base of the atrial appendage where it joins the main atrial chamber, thereby closing off the appendage to the flow of blood. While effective, this technique generally requires an open chest procedure, i.e., open heart surgery, as suturing an appendage closed is very difficult to perform in a closed-chest environment and is generally not attempted.

Other techniques that have been used include: placing a line of staples across the base of the appendage, or filling the appendage with a space occupying device to fill up the cavity otherwise available for blood to flow into, in an effort to prevent blood flow into the atrial appendage cavity, and ultimately, to prevent blood clot formation there. Staplers have been used in closed-chest procedures for atrial appendage exclusion. Endoscopic gastrointestinal anastomotic (GIA) staplers are what are presently used to perform closed chest left atrial appendectomy. A GIA stapler is used to place one or more lines of staples across the base of the appendage. However, difficulties present with use of this technique, as there is a tendency for the staples to tear into the friable tissue of the appendage and cause bleeding, requiring the chest to be opened to repair the damage to the torn appendage. Further, since staplers that are presently used for these procedures are not designed for use on an atrial appendage, but rather for gastrointestinal use, the closure force on the staples, as the staples are placed in the appendage, may not be suitable for the tissue to which the force is applied. Further, the tissue thickness of the walls of the appendage may differ significantly from tissue thicknesses that the stapler is designed to close, resulting either in tissue damage to the appendage by the applied staple drawing the tissue walls too close together and thus crushing them, or incomplete closure, resulting in a failure to completely close off the appendage to the flow of blood. Still further, a line of staples placed may leave small pouches of atrial appendage at each end of the staple line. These residual pouches may be a source of thrombus (clot) formation.

Space occupying devices that are currently used also tend to leave areas of the appendage exposed to the blood path (circulation), with potential thrombus formation, and are particularly susceptible to this when delivered under closed chest conditions, such as via catheter, for example.

There is a continuing need for techniques and devices for excluding an atrial appendage (left and/or right atrial appendage) using minimally invasive procedures (e.g., closed chest procedures). Techniques that do not require a median sternotomy or substantial thoracotomy would decrease morbidity as well as hospitalization time.

### SUMMARY OF THE INVENTION

Devices, tools and methods for occluding fluid flow between two walls of tissue in a patient. Two walls of tissue are compressed together with sufficient compressive force to prevent fluid flow between the two walls, while ensuring that the compressive force is not so great as to cause tissue necrosis. The devices, tools and methods may be carried out using minimally invasive surgical techniques, such as in reduced-access surgical sites, including, but not limited to delivery, via a subxyphoid minimal incision.

Devices, tools and methods are provided for occluding an atrial appendix.

In at least one embodiment, a device for occluding fluid flow between two walls of tissue in a patient is provided, including: a first jaw configured to apply compressive force against a first of the two walls; and a second jaw configured to apply compressive force against a second of the two walls upon installing the device, wherein, when installed, said first and second jaws compress the two walls therebetween. The first and second jaws have an open configuration, in which first end portions thereof are joined by a joint and second end portions thereof are separated. The first and second jaws are movable to a closed configuration in which the first end portions are joined by the joint and the second end portions are connected by an automatic locking mechanism.

In at least one embodiment, a closure driver is mechanically connected to the first and second jaws, and is actuatable from a location outside of a patient, to move the first and second jaws from the open configuration to the closed configuration when the device is located internally of the patient.

In at least one embodiment, a tool used to deliver a device according to the present invention includes an elongate shaft having a proximal and distal portion where the distal portion of the shaft movably mates with the device. The device is implantable and includes an elongate body extending between a proximal and distal portion, and can include two opposed clamping members sized and shaped to receive at least a portion of a left atrial appendage therebetween, which is typically a left atrial appendage of a human, although not necessarily limited thereto. The proximal portion of the device can include a mating feature for detachably mating with a distal portion of the tool. In addition, the implantable device can comprise a distal hinge connecting the two clamping members and a proximal locking mechanism for locking the two clamping members relative to one another. In one aspect, an assembly also includes an articulating mechanism extending along at least a portion of the tool for moving the implantable device relative to a shaft of the tool.

In at least one embodiment, the articulating mechanism can be operated to move the device between an insertion configuration, in which the assembly has a low-profile configuration, and a clamping configuration, in which the device is moved into position for approaching and/or clamping target tissue. For example, the articulating mechanism can pivot the device relative to a shaft of the tool to move between the insertion configuration and the clamping configuration. In one aspect, the device extends distally from a distal end portion of the tool and/or extends along an axis defined by at least a portion of an elongate shaft of the tool when the device is in the insertion configuration. In the clamping configuration, the device is positioned at an angle with respect to the elongate shaft of the tool and/or at an angle with respect to the position of the device in the insertion configuration.

In at least one embodiment, the connection between the device and the tool allows relative movement of the device with respect to the tool and allows the device to detachably connect to the tool. In addition, the assembly can further comprise a second detachable connection, the second detachable connection being between an articulation mechanism of the tool and the device. To detach and implant the device, the connection between the device and a shaft of the tool and the connection between the device and the articulation mechanism can be detached.

In at least one embodiment, an assembly is provided that includes a tool having first and second elongate shafts. The first elongate shaft can extend between a proximal and distal end and include a first mating surface for movably mating with a implantable device. The second elongate shaft can extend between a proximal and distal end and include a second mating surface. The assembly can further comprise an implantable device having first and second device bodies with proximal and distal ends and a pivot point connecting the two device bodies proximate to their distal ends. The proximal end of the device can include a locking mechanism for locking the device bodies to one another. The implantable device can further comprise a first device mating surface positioned on the first device body for detachable mating with the first mating surface of the first shaft, and a second device mating surface positioned on the second device body for detachably mating with the second mating surface of the second shaft.

In at least one embodiment, the implantable device comprises an implantable clip.

In at least one embodiment, movement of the first shaft relative to the second shaft moves the device with respect to the second shaft. Additionally, or alternatively, movement of the first shaft relative to the second shaft can control opening and/or closing of the implantable device.

In at least one embodiment, in addition to the first and second shafts being detachably mateable with the implantable device, the shafts and implantable device are movably mated. For example, the detachable connections can allow the shafts to pivot, rotate, and/or translate relative to the implantable device.

An implantable device is disclosed that comprises an elongate clamp body extending between a proximal end and a distal end and including first and second clamping members having first and second opposing surfaces. The clamping members can be movably mated with one another proximate to the distal end of the clamp body. The clamp can further comprise a locking mechanism proximate to the proximal end of the clamp body for locking the first and second clamping members to one another, and a first and a second mating surface for detachably mating the clamp body to a delivery tool.

In at least one embodiment, the locking mechanism extends from the first opposing face of the first clamping member. For example, the locking mechanism can extend through an aperture in the second clamping member when the first and second clamping members are locked.

In at least one embodiment, the implantable device comprises a tissue barrier adapted to inhibit pinching of tissue in the locking mechanism. In at least one embodiment, the tissue barrier extends between the first and second clamping members. The tissue barrier can be positioned such that when target tissue is positioned between the first and second clamping members the tissue barrier separates the tissue to be clamped from the locking mechanism. In at least one embodiment, the tissue barrier is positioned proximate to the proximal end of the clamp body. In at least one embodiment, the tissue barrier mates with the first opposing surface of the first clamping member and extends through an aperture in the second clamping member.

A device for occluding fluid flow between two walls of tissue in a patient is provided, including: a first jaw configured to apply compressive force against a first of the two walls; and a second jaw configured to apply compressive force against a second of the two walls upon installing the device, wherein, when installed, the first and second jaws compress the two walls therebetween. The first and second jaws have an open configuration, in which first end portions of the first and second jaws are joined by a joint and second end portions of the first and second jaws are separated. The first and second jaws are movable to a closed configuration in which the first end portions are connectable. A closure driver, mechanically connected to the first and second jaws, is actuatable from a location outside of a patient, to move the first and second jaws from the open configuration to the closed configuration when the device is located internally of the patient.

In at least one embodiment, the implantable device can further include a tissue barrier extending between the two clamping members or jaws. For example, a tissue barrier can be positioned adjacent to the device locking mechanism to inhibit pinching of the clamped tissue in the locking mechanism

A tool for minimally invasive delivery and installation of an occlusion device is provided, including: an elongated shaft connecting distal and proximal end portions, the tool being configured and dimensioned to deliver the distal end portion through a small opening in a patient, to a reduced-access surgical location, while the proximal end portion of the tool remains outside of the patient. The distal end portion includes a platform configured to releasably engage the occlusion device. The proximal end portion includes a release actuator actuatable from outside of the patient, to release the device from the distal end portion located in the reduced-access surgical location inside the patient.

An assembly is provided, including: a device releasably mounted to a distal end portion of a tool, the device and tool configured and dimensioned for delivery of the device through a minimally invasive opening in a patient to a target surgical site, while a proximal end portion of the tool remains outside of the patient, for occluding fluid flow between two walls of tissue in a patient. The device includes a first jaw configured to apply compressive force against a first of two walls of tissue to be compressed together, and a second jaw configured to apply, compressive force against a second of the two walls upon installing the device. The first and second jaws are connected at first end portions thereof by a joint. The tool includes an elongated shaft connecting distal and proximal end portions thereof. The distal end portion of the tool includes a platform configured to releasably engage the device. The proximal end portion of the tool includes a release actuator actuatable from outside of the patient, to release the device from the distal end portion of the tool when located in the target surgical site inside the patient. The device is releasably connected to the platform.

Methods of implanting a device according to the present invention are provided. In at least one embodiment, a method includes: providing an assembly comprising an implantable device having an elongate body that is movably mated with an elongate shaft and inserting the implantable device through a surgical opening while the device is positioned in a low profile configuration. After insertion, the method can include moving the implantable device relative to the shaft to position the device for clamping, and clamping tissue between clamping members of the implantable device. The device can then be detached from the elongate shaft, and the shaft can be removed from the patient. In one embodiment, the device is inserted via a subxyphoid approach. In another embodiment, the device can be inserted through a left or right side approach, such as, for example, through a left or right side port.

In at least one embodiment the moving of the implantable device relative to the shaft includes pivoting the implantable device relative to the shaft. The moving and/or detaching of the device can be controlled via controls positioned at or near the proximal end portion of the shaft.

A method of performing an occlusion of fluid flow between two walls of tissue in a patient is provided, including: inserting an occlusion device connected to a tool through a minimally invasive opening in a patient; delivering the occlusion device to a location of the two walls to be occluded; positioning opposite jaws of the device against the two walls, respectively; clamping the walls between the jaws by closing and locking the jaws; and releasing the device from the tool.

These and other features of the invention will become apparent to those persons skilled in the art upon reading the details of the devices and methods as more fully described below.

### BRIEF DESCRIPTION OF THE DRAWINGS

The accompanying drawings, which are incorporated in and constitute a part of this specification, illustrate exemplary embodiments of the invention and together with the description, serve to explain the principles of the invention.

FIG. 1A is a partial side view of an assembly including an occlusion device and delivery tool according to an exemplary embodiment of the present invention.

FIG. 1B is a partial perspective view of another embodiment of assembly according to the present invention.

FIG. 2 is a partial perspective view of the assembly illustrated in FIG. 1A.

FIG. 3 is a partial perspective view of the assembly illustrated in FIG. 2 showing an open implantable device.

FIG. 4A is an exploded partial view of the assembly illustrated in FIG. 2.

FIG. 4B is a partial perspective view illustrating a wire or filament extending through the actuating rod shown in FIG. 4A.

FIG. 5 is a bottom partial view of an assembly including one embodiment of a delivery tool according to the present invention.

FIG. 6 is a bottom partial view of the assembly of FIG. 5 in an unlocked configuration.

FIG. 7 is a side partial view of the assembly illustrated in FIG. 6.

FIG. 8 is a side view of an exemplary embodiment of an implantable device described herein.

FIG. 9 is a perspective partial view of another exemplary embodiment of an implantable device described herein.

FIG. 10 is a perspective view of an occlusion device according to the present invention, the occlusion device being shown in a closed configuration.

FIG. 11 illustrates a device mounted to a distal end portion of a tool, with the device being shown in an open configuration.

FIG. 12 illustrates a mechanical linkage releasably linking a device to a tool.

FIG. 13 is a partial perspective view of a tool having an occlusion device connected thereto.

FIG. 14 illustrates a device having been installed near the base of an atrial appendage to occlude the atrial appendage.

### DETAILED DESCRIPTION OF THE INVENTION

Before the present devices, tools, assemblies and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a latch” includes a plurality of such latches and reference to “the hinge” includes reference to one or more hinges and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

The term “open-chest procedure” refers to a surgical procedure wherein access for performing the procedure is provided by a full sternotomy or thoracotomy, a sternotomy wherein the sternum is incised and the cut sternum is separated using a sternal retractor, or a thoracotomy wherein an incision is performed between a patient's ribs and the incision between the ribs is separated using a retractor to open the chest cavity for access thereto.

The term “closed-chest procedure” or “minimally invasive procedure” refers to a surgical procedure wherein access for performing the procedure is provided b, one or more openings which are much smaller than the opening provided by an open-chest procedure, and wherein a traditional sternotomy is not performed. Closed-chest or minimally invasive procedures may include those where access is provided b, any of a number of different approaches, including mini-sternotomy thoracotomy or mini-thoracotomy, or less invasively through a port provided within the chest cavity of the patient, e.g., between the ribs or in a subxyphoid area, with or without the visual assistance of a thoracoscope.

The term “reduced-access surgical site” refers to a surgical site or operating space that has not been opened fully to the environment for access by a surgeon. Thus, for example, closed-chest procedures are carried out in reduced-access surgical sites. Other procedures, including procedures outside of the chest cavity, such as in the abdominal cavity or other locations of the body, may be carried out as reduced access procedures in reduced-access surgical sites. For example, the surgical site may be accessed through one or more ports, cannulae, or other small opening(s). What is often referred to as endoscopic surgery is surgery carried out in a reduced-access surgical site.

### Devices, Tools, Assemblies and Methods

Atrial appendage management, and particularly left atrial appendage (LAA) management, is a critical part of the surgical treatment of atrial fibrillation. When using a minimally invasive approach (e.g., where surgical access is provided by thoracoscopy, mini-thoracotomy or the like), there is a high risk of complications such as bleeding, when using contemporary atrial appendage management, as noted above. Further, exposure and access to the base of the atrial appendage to be treated is limited by the reduced-access surgical site. The present invention provides devices, tools assemblies and methods for ligating or occluding an atrial appendage, which ligation or occlusion may be performed while the heart continues to beat, and wherein such ligation or occlusion methods may be preformed using a minimally invasive approach. Such procedures may be performed solely from an opening in the right chest, or may be performed from a single opening in the left chest, or from a small sub-xyphoid opening, for example, if desired by the surgeon performing the procedure. For example, the opening through which the devices of the present invention may be inserted may be, a port or trocar commonly used in endoscopic surgical procedures. Particular locations in which small incisions may be made through which to deliver a device to perform atrial appendage ligation include, but are not limited to: the left third or fourth intercostal space, the right third or fourth intercostal space, or a subxyphoid location.

Described herein are various methods, assemblies, tools and devices for clamping tissue, particularly cardiac tissue. In one aspect, an assembly for delivering an implantable device includes an implantable occlusion device and an elongate shaft. The implantable device can extend from a distal portion of the shaft such that the combined device and shaft have a low-profile configuration. In use, this low profile configuration permits implantation of the device with minimal patient trauma. In another aspect, the low profile configuration of the assembly permits implantation via a sub, phoid approach to the left atrial appendage. The assembly can further comprise a movable connection between the shaft and device to allow at least a portion of the device to move relative to at least a portion of the shaft. In one aspect, the movable connection allows the device to move from a low profile insertion configuration to a clamping configuration and/or to a device implantation configuration. Additionally, the assembly can include a detachable connection that permits all or a portion of the device to detach from the shaft, so that the device can be detached and implanted after clamping tissue.

In at least one embodiment, the implantable device is configured for clamping at least a portion of the left atrial appendage. For example, the device can be formed in a size and shape commensurate with the left atrial appendage and the body cavity in which the left atrial appendage is located. For example, the device can comprise a clip. The device can include at least two opposable clamping members and a locking mechanism for fixing the opposable clamping members relative to one another. In one aspect, the space between the two opposable clamping members, when fixed via the locking mechanism, is sized and shaped to receive a portion of the left atrial appendage. However, while the implantable device is described herein with respect to clamping the left atrial appendage, one skilled in the art will appreciate that the assemblies, tools, methods, and devices described herein can be configured for clamping other anatomical features.

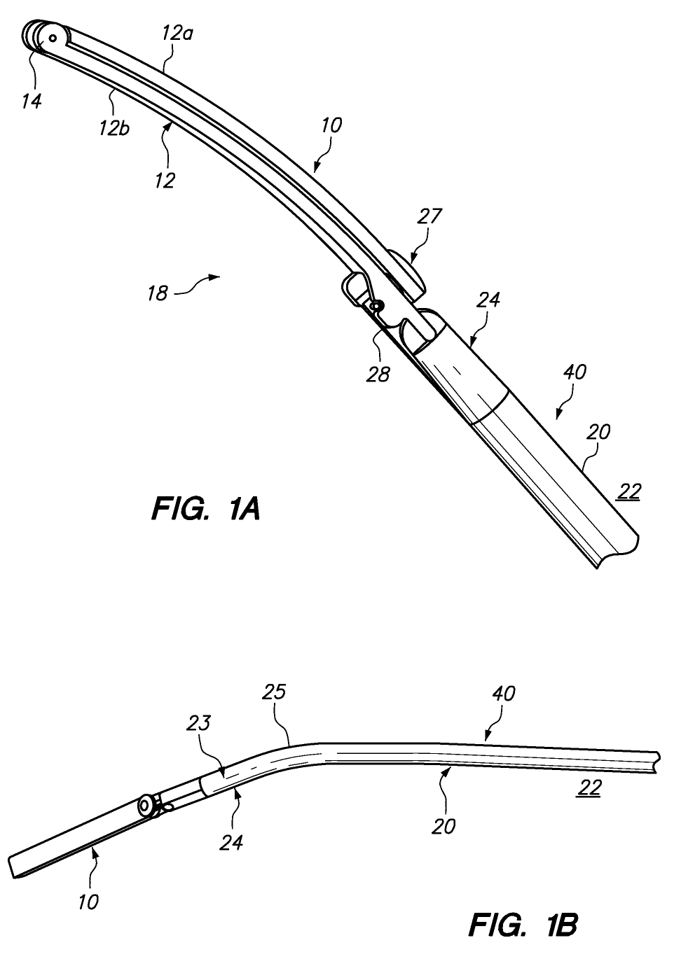

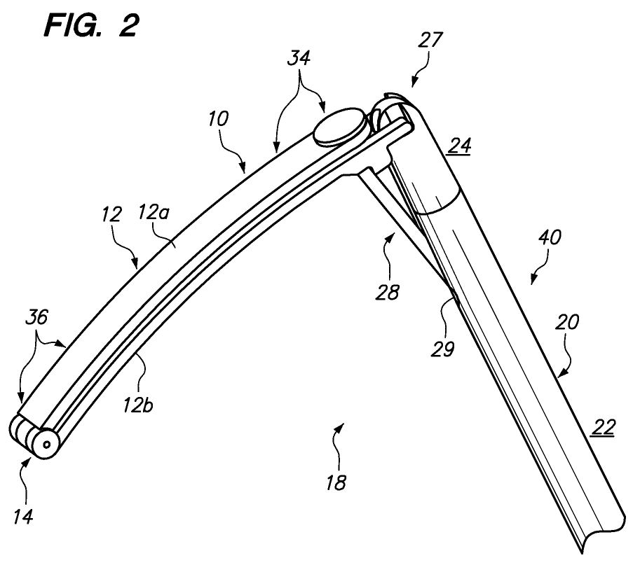

The tools described herein enable delivery, e.g., insertion and implantation, of a device via minimally invasive procedures. FIGS. 1A and 2 illustrate one exemplary embodiment of an assembly 18 with device 10 shown in an insertion configuration (FIG. 1A) and in an implantation configuration (FIG. 2). An elongate shaft 20 of tool 40 can be configured to allow implantation of device 10 at a distance. In one aspect, shaft 20 can include a generally single piece structure with device mating features positioned proximate to the distal end portion 24 of shaft 20. Alternatively, shaft 20 can be formed from multiple pieces fixedly or detachably mated with one another, thereby enabling a user to configure the assembly 18 for use with patients having chest cavities of different sizes and permitting implantation of the device 10 over a wide range of distances. Furthermore, as discussed in more detail below, a portion of shaft 20 can be detached to disconnect device 10 from shaft 20. Alternatively, or additionally, a movable connection between shaft 20 and device 10 can be detachable. For example, detachable connection 27 between shaft 20 and device 10 can permit relative movement between shaft 20 and device 10, as well as, detachment of device 10 from shaft 20. In still another embodiment, the movable connection between device 10 and shaft 20 can be separate from a detachable connection between device 10 and shaft 20.

Generally, shaft 20 can be made of surgical grade materials including metals, polymers, ceramics, composites, and combinations thereof, such as, for example, stainless steel or other metals or alloys. The elongate shaft 20 can be formed from any sufficiently rigid type of material that can be subjected to mechanical forces sufficient to insert the system into the body (i.e., pushing, pulling and/or twisting along an axis) and to open and close the implantable clip. The material of the elongate shaft 20 can be designed for single use or for multiple uses. If designed for multiple uses, it can be fabricated from materials designed to withstand sterilization by radiation, conventional autoclaving at high temperature and pressure, and/or other similar procedures for sterilization of surgical tools, instruments, sutures, or other medical implements intended for use inside the body.

Shaft 20 can have an elongate shape extending between a proximal end 22 and a distal end 24. In one aspect, shaft 20 extends along a longitudinal axis and has a generally linear appearance. Alternatively, shaft 20 can be non-linear or have a curved segment. The shape of shaft 20 can be chosen based on a variety of factors including, for example, the intended use of assembly 18, the target tissue to be clamped, the incision location, and/or the size and shape of anatomic structure (e.g., the size, shape, and/or relative location of a body cavity). In one aspect, illustrated in FIG. 1B, shaft 20 includes a linear distal portion 23 with a bend 25 positioned proximally thereof. In the insertion configuration, device 10 can extend along an axis defined by a portion of shaft 20, such as distal portion 23.

Assembly 18 can be configured to move at least a portion of device 10 from an insertion configuration in which assembly 18 has a low-profile (e.g., a narrow width or relatively small cross sectional profile over the length thereof) to a clamping or implantation configuration in which device 10 is orientated for clamping target tissue (e.g., the LAA). In one embodiment, while in the insertion configuration, the elongate body of device 10 extends distally from the distal portion 24 of shalt 20. For example, the device body can extend along an axis substantially co-linear, co-axial, and/or parallel to a portion of shaft 20. Actuation of assembly 18 can move assembly 18 into the implantation configuration such that device 10 is positioned at an angle relative to elongate shaft 20 and/or relative to the position of device 10 in the insertion configuration.

In one aspect, an actuating mechanism extends along at least a portion of shaft 20. In one exemplary embodiment, the actuating mechanism includes an actuating rod 28, although other force transmitting elements known to one of ordinary skill in the mechanical and surgical arts, such as pull wires, gears, and the like, are also contemplated. In the exemplary embodiments illustrated in FIGS. 1A-3, an actuating rod 28 extends through a lumen 29 within elongate shaft 20 and detachably mates with device 10. User translation (i.e., pushing, pulling, and/or rotation) of actuating rod 28 moves the implantable device 10 between the insertion configuration (see, e.g., FIG. 1A) and the implantation configuration in which the implantable device 10 is positioned at an angle with respect to elongate shaft 20 of tool 40 (see, e.g. FIG. 2).

The low-profile arrangement of the implantable device 10 with respect to the elongate shaft 20 in the insertion configuration facilitates deliver), of the implantable device 10 through a small incision. For example, device 18 can be inserted through a small endoscopic or laparoscopic incision via a trocar, cannula, or other similar surgical device.

In certain embodiments, and as illustrated in FIG. 2, the implantable device 10 comprises a first clamping member or jaw 12a and a second clamping member or jaw 12b extending between proximal and distal ends. The proximal end portion 34 of the implantable device 10 can include a first mating surface for detachably mating with the distal portion 24 of elongate shaft 20, and a locking mechanism as discussed in more detail below. The distal end portion 36 of implantable device 10 comprises a pivot point or hinge 14 connecting the first 12a and second 12b clamping members/jaws. Additional features of the implantable device 10 are discussed in more detail below.

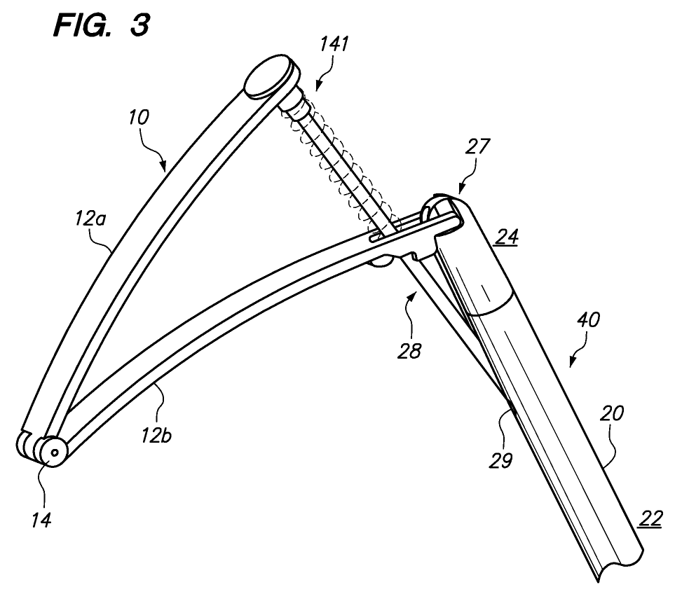

After a portion (including device 10 and part of tool 40) of assembly 18 in a low-profile insertion configuration has been inserted, for example, into a patient's chest cavity, and guided into position adjacent to the tissue that is desired to be occluded (e.g., the LAA), a user can then pivot device 10 from the insertion configuration to the implantation configuration using an actuating mechanism such as actuating rod 28, as illustrated in FIGS. 2-3. Once device 10 is in an implantation configuration, as shown FIG. 2: the user can then unlock and open device 10. With device 10 open, the user can guide device 10 over the tissue to be occluded and then close and lock device 10.

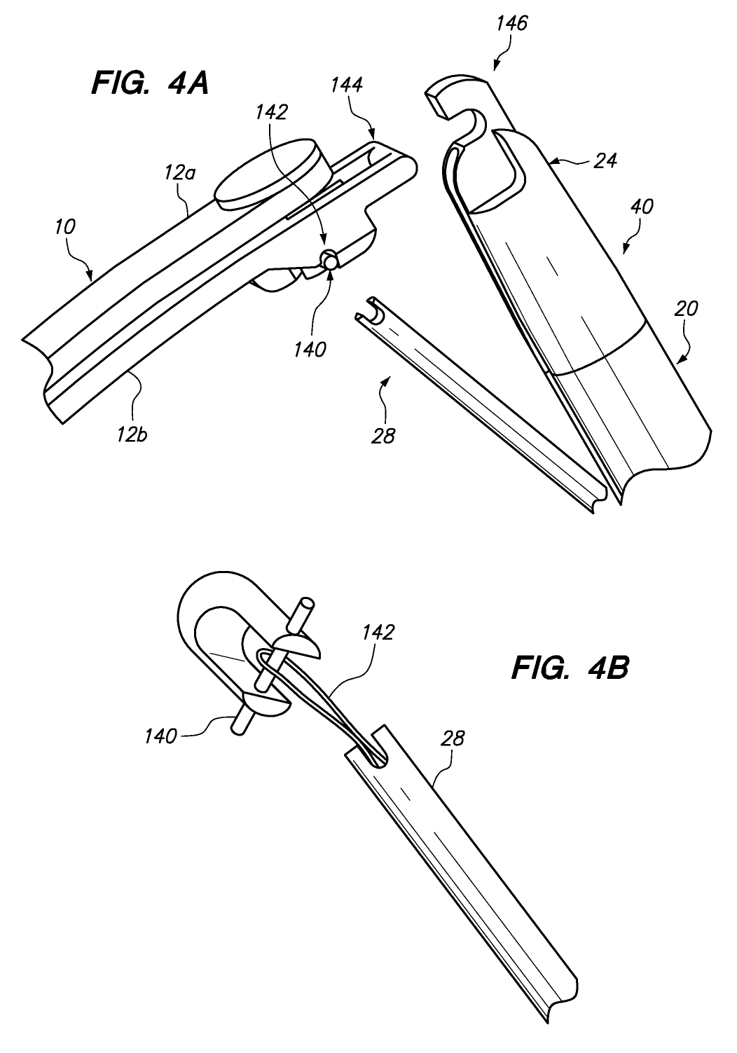

After delivery of device 10, a user can detach device 10 from elongate shaft 20 before removing shaft 20 from the body cavity and closing the incision. The detachable connection between device 10 and shaft 20 can be defined by a portion of device 10, shaft 20, and/or another member mated with device to and/or shaft 20. For example, a portion of shaft 20 and/or device 10 can be designed to break-away or detach and allow separation of device 10 and shaft 20. Alternatively, the detachable connection can be defined by the connection between device 10 and shaft 20. In one such embodiment, the connection between device 10 and shaft 20 allows relative movement between device 10 and shaft 20 (e.g., movement between an insertion configuration and a clamping configuration), as well as detachment of device 10 from shaft 20. For example, as illustrated in FIG. 4A, distal end portion 24 of elongate shaft 20 can include a “C”-shaped hook 146 having a surface shape generally corresponding to the shape of a bar 144 positioned on device 10. Bar 144 can rotate relative to hook 146 to allow device 10 to move relative to shaft 20. To detachably secure implantable device 10 to distal portion 24 of elongate shaft 20, a user places bar 144 into hook 146 and rotates hook 146 counterclockwise, thereby reversibly engaging implantable device 10 and elongate shaft 20 of tool 40. To detach device 10, hook 146 can be rotated by a controller positioned at the proximal end of device via, for example, a control wire, push rod, and/or gears. While the mating surfaces of device 10 and shaft 20 are illustrated as a hook 146 and bar 144, respectively, other mechanical and/or frictional mating features are also contemplated.

In at least one embodiment, implantable device 10 can also detachably mate with an actuating mechanism, such as hollow actuating rod 28, via a second detachable connection 141, as shown in FIG. 3. In one aspect, device 10 includes a mating surface for detachably mating with rod 28. For example, in the exemplary embodiment of FIGS. 2 through 4B, hollow rod 28 passes through lumen 29 of elongate shaft 20 and mates with device 10. In one aspect, actuating rod 28 can move device 10 between an insertion configuration and a clamping configuration. For example, manipulating or translating actuating rod 28 causes device 10 to pivot at the detachable connection 27 between device 10 and shaft 20, thereby moving assembly 18 between the low-profile insertion configuration (FIG. 2) and the implantation configuration (FIG. 3). In addition, or alternatively, movement of rod 28 can open first and second members/jaws 12a, 12b relative to one another, to control clamping. Once in the clamping configuration, the connection between actuating rod 28 and device 10 can enable a user to unlock and open implantable device 10 before occluding the LAA. That connection also permits a user to close and lock implantable device 10 before detaching the implantable device 10 and removing elongate shaft 20 following successful completion of the LAA occlusion procedure.

In at least one embodiment, a detachable connection between rod 28 and device 10 includes a pin 140 (FIGS. 4A through 7). The distal end of rod 28 can include a “U” shaped opening or recess in which pin 140 sits. In one aspect, the “U” shaped opening and pin 140 are sized and shaped to allow movement between pin 40 and rod 28. For example, rod 28 can rotate relative to pin 40.

In at least one embodiment, a wire or filament 142 can extend through rod 28 and over pin 140 to detachably mate rod 28 and device 10, see FIG. 4B Cutting or cutting and withdrawing the wire or filament 142 allows rod 28 to detached from pin 40. Cutting can be performed with standard surgical scissors, for example. While a rod 28 and pin 140 configuration is illustrated as one method of connecting the device 10 and rod 28, other detachable connections, such as threads or a clasp are also contemplated.

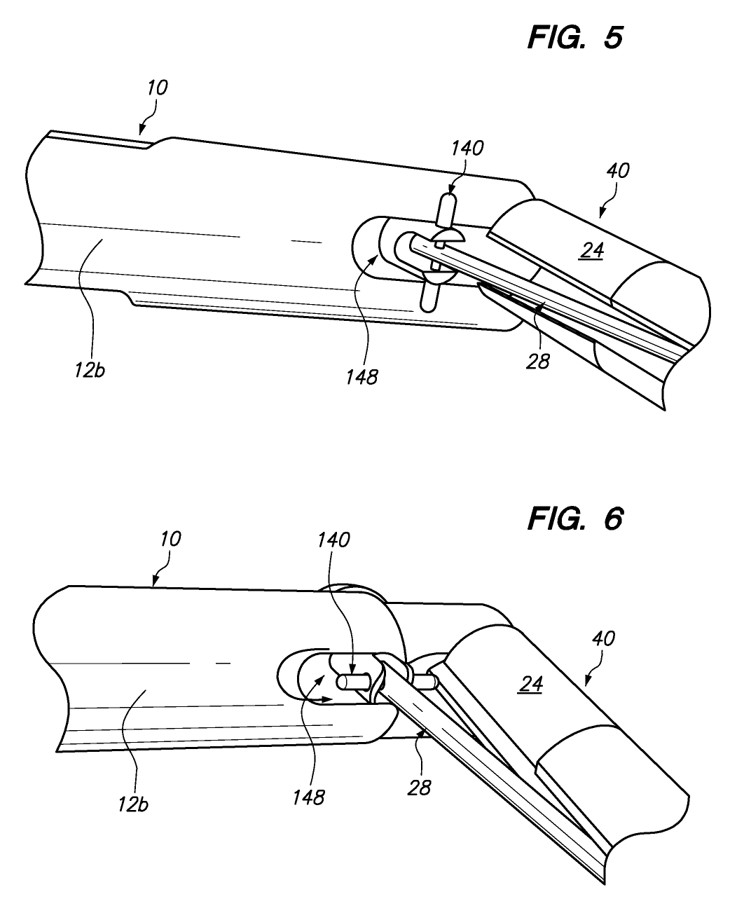

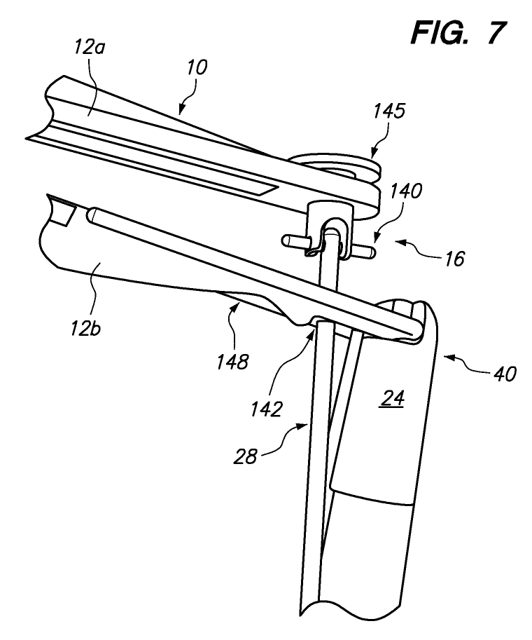

Implantable device 10 can further comprise a locking mechanism 16 for locking the first and second members 12a, 12b of device 10 to one another. In certain embodiments, pin 140 defines part of the locking mechanism 16. For example, as illustrated in FIG. 7, pin 140 can include a body 145 that rotatably mates with first member/jaw 12a. The connection between pin body 145 and device 10 allows pin 140 to rotate relative to first and second members 12a, 12b. To lock the first and second members 12a, 12b relative to one another and thereby prevent opening or closing movements of the members 12a, 12b relative to one another, rotatable pin 140 passes through a corresponding slot 148 in second clamping member/jaw, 12b, see, e.g., FIGS. 5 and 6. Slot 148 can include a first dimension that allows passage of pin 140 and a second dimension that is smaller than the length of pin 140. After passage of pin 140 through slot 148, pin 140 can be rotated into a configuration which prevents passage of pin 140 back through slot 148. For example, when pin 140 is oriented perpendicularly to the elongate body of implantable device 10 (FIG. 5), the width of slot 148 can prevent the passage of pin 140 and thereby lock first and second members 12a, 12b. In addition, rotatable pin 140 can be seated in a corresponding notch 142 or other surface feature on second clamping member 12b to inhibit unlocking of the first and second members 12a, 12b. One skilled in the art will appreciate that other locking mechanisms 16 can be substituted for the illustrated pin 140/slot 148 connection and, in certain embodiments, the locking mechanism 16 of the implantable device 10 can further comprise an adjustable closure that can be tightened or loosened incrementally depending on the thickness of the tissue to be placed between clamping members 12a, 12b, such as a screw or other type of adjustable fastener known to one of skill in the art.

Like shaft 20, implantable device 10 can be manufactured in a variety of sizes for use with patients of different ages and/or physical sizes. One of ordinary skill in the art could determine the appropriate size of implantable device 10 for a particular patient by applying standard diagnostic criteria well known in the medical and surgical arts. Implantable device 10 can be made of surgical grade materials including metals, polymers, ceramics, composites, and combinations thereof, such as, for example, stainless steel or other metals or alloys. Implantable device 10 can also be formed from any sufficiently rigid type of material that can be subjected to mechanical forces sufficient to insert the assembly into the body (i.e., pushing, pulling and/or twisting along an axis) and to open and close implantable device 10. Device 10 can also be fabricated from materials designed to withstand sterilization by radiation, conventional autoclaving at high temperature and pressure, or other similar procedures for sterilization of surgical tools, instruments, sutures, or other medical implements intended for use inside the body cavity.

In certain embodiments, the distal end portion 36 of implantable device 10 comprises a hinge 14 connecting the first 12a and second 12b clamping members, see FIGS. 2 and 3. Hinge 14 may be a traditional mechanical hinge, a living hinge, or any other type of flexible hinged connection known to one skilled in the mechanical or surgical arts. The term “living hinge,” as used herein, refers to a hinge or flexure bearing with no moving parts. A living hinge comprises a thin section of material that bends to allow movement, such as the lid on a box of TIC-TAC® mints (breath mints) or other disposable packaging.

In one aspect, the first and second clamping members 12a, 12b can be biased in an open or closed configuration. For example, a spring or resilient material (biasing member 143) can optionally be provided to bias device 10 in the open configuration (illustrated in phantom lines in FIG. 3). After unlocking device 10, the bias provided by the biasing member 143 against clamping members 12a, 12b can cause device 10 to open if not restrained by rod 28.

First 12a and/or second 12b clamping members can be formed to include a depression or groove 154 suitable to accommodate LAA tissue. In addition, one or both of the clamping members 12a, 12b may be lined with a compressible or flexible material 18 to improve grip or the ability to hold tissue in place after the implantable device 10 is closed, locked and implanted. In addition, or alternatively, clamping members 12a, 12b can include a high friction surface or surface feature to assist with clamping tissue. For example, ridges and/or recesses can be positioned along the contact surfaces 150, 152 of clamping members 12a, 12b configured to contact the tissue upon actuating the clamping action.

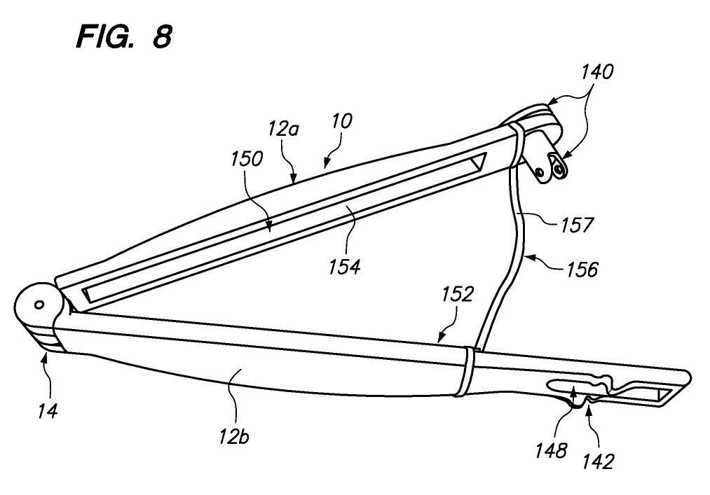

In one aspect, illustrated in FIG. 8, at least one of the opposing inner surfaces (i.e., contact surfaces) 150, 152 of first and second members 12a, 12b can include an elongate recess or groove 154 for receiving clamped tissue. For example, a longitudinal channel 54 extending between the sidewalls of first member 12a can trap and hold tissue when first and second members 12a, 12b 32 are clamped together.

In at least one embodiment, implantable device 10 further comprises a tissue barrier 156 to direct tissue away from the device locking mechanism 16 and to reduce the chance of tissue being pinched within the locking mechanism 16 when first and second clamping members 12a, 12b are locked to one another. In one embodiment, tissue barrier 156 is defined by a band 157 extending between the first 12a and second 12b clamping members. As the first and second members 12a, 12b converge to clamp tissue, band 157 inhibits entry of LAA tissue into the locking mechanism 16 and thereby facilitates closure and locking of device 10. In one aspect, band 157 is flexible and/or stretchable. The opening movement of device 10 can expand (e.g., stretch/extend) band 157, while the closing of device 10 allows the flexible band 157 to return to its original configuration. In instances where band 157 is stretchable by being elastic, opening of device plastically deforms band 157 as it elastically elongates, and upon closing of device 10, the elasticity or band 157 returns it to its undeformed starting length.

Band 157 can have a width equal to or greater than rod 28 and/or slot 48. In one embodiment, band 157 has a width approximately equal to the width of adjacent first and/or second clamping member 12a, 12b. As the first and second clamping members 12a, 12b converge, the width of band 157 inhibits entry of the band 147 into slot 148 and/or pinching of band 157 in device locking mechanism 16.

In at least one embodiment, tissue barrier 156 mates with first and second members 12a, 12b proximate to the proximal end of device 10 and/or adjacent to device locking mechanism 16. In another embodiment, tissue barrier 156 can be mated with rod 28 in addition to, or as an alternative to, mating to second member 12b and/or first member 12a

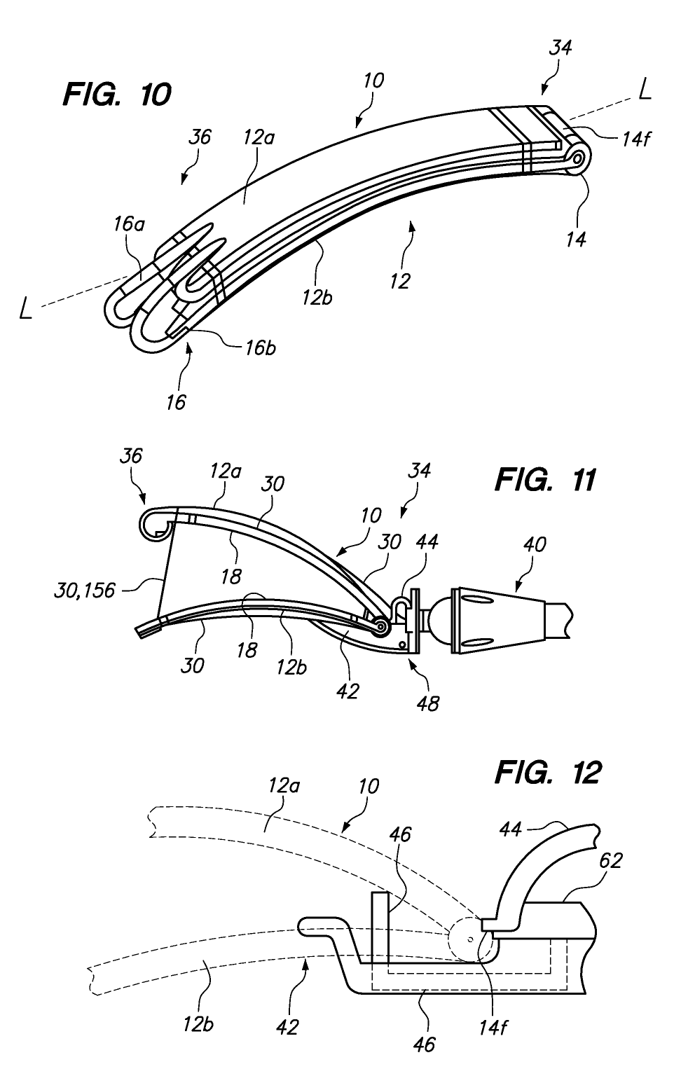

In another embodiment, tissue barrier 156 may be attached to implantable device 10 in such a way that a user can maintain tension on the tissue barrier 156 as implantable device opens/closes. For example, a pull wire can extend to tissue barrier 156 The closure driver 30 in the embodiment of FIGS. 10-14 also function as a tissue barrier 156 in this manner.

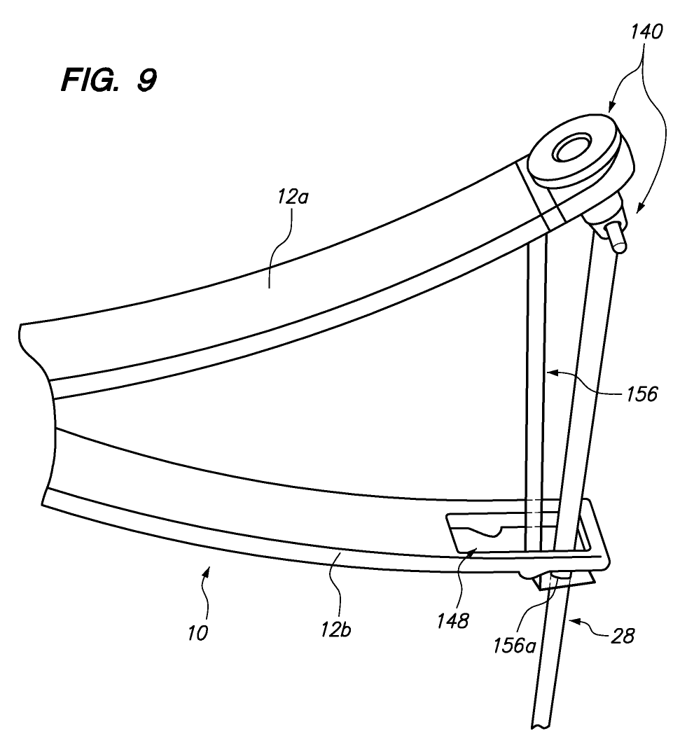

In still another embodiment, the tissue barrier can be defined by a rigid or semi-rigid member. As illustrated in FIG. 9, a rigid tissue barrier 156 can move through slot 148 as the first and second members 12a, 12b converge. For example, the rigid tissue barrier 156 can mate with rod 28 such that tissue barrier 156 moves with rod 28 as rod 28 traverses slot 148. In one aspect, rigid tissue barrier 156 movably mates with rod 28 to allow rod 28 to rotate relative to tissue barrier 156. For example, rod 28 can rotate relative to tissue barrier 156 while performing locking/unlocking of device 10. As illustrated in FIG. 9, rigid tissue barrier 156 can include an aperture 156a through which rod 28 passes. A recess and/or protrusions on rod 28 can allow rod 28 to rotate within tissue barrier 156, while prohibiting relative longitudinal movement between rod 28 and tissue barrier 156.

Referring now to FIG. 10, another embodiment of a device 10 for occluding an atrial appendage is shown. Device 10 in this example comprises a device that is configured to close over the base portion of the left atrial appendage to close off the atrial appendage to the flow of blood. Device 10 may come in a variety of dimensions to accommodate variations in the size of the atrial appendage base to be ligated. Device 10 may also be used to ligate the right atrial appendage, and the variations in dimension of device 10 may be advantageous to expand the range or tissues that may be ligated by device 10.

In this example, device 10 includes has an atraumatic contour to allow it to rest against the heart naturally when it is clipped in place over the left atrial appendage or so as to be atraumatic to other surrounding tissues when it is clipped over some other tissue. The atraumatic contour includes gently curved or rounded ends and other components. Additionally, the main body of device 10 is curved so that ends of the main body extend out of a plane in which the longitudinal axis resides. This curvature generally matches the curvature of the heart adjacent the base of the left atrial appendage, so that when device 10 is implanted, it rests with conforming contact to the surface of the heart. Device 10 includes a clip frame 12 having first and second members, portions or jaws 12a, 12b joined by a hinge 14 at one end of device 10. Jaws 12a and 12b may be made of a rigid material, or ma) be malleable to allow shaping, or somewhat flexible, as long as enough rigidity is retained to maintain the shapes of the jaws 12a,12b when the), are clamped closed against tissue surfaces, so as to maintain a clamping action against the tissues without substantially deforming, thereby preventing fluid flow between the walls of the tissue clamped.

Locking mechanism 16 is provided at an end of device 10 opposite the end at which hinge 14 is formed, which, in the example of FIG. 10 is at the distal end portion 36 of device 10 when installed in assembly 18, as illustrated in FIG. 11. Locking mechanism 16 may be formed from tabs 16a that are each fixed at one end to an end portion of one of the jaws (jaw 12a in the embodiment shown in FIG. 10). The tabs 16a extend longitudinally away from the end of the jaw 12a that they are attached to and in a closed configuration, as shown in FIG. 10, the free end of each tab 16a is directed toward the other jaw (in this example, jaw 12b). The other jaw includes mating features 16b, such as relatively rigid tabs or other relatively rigid protrusions that deflect the free ends of tabs 16a as they are driven past the free ends during closing the device 10. The free ends then resiliently return to their undeflected configurations and capture the features 16b, thereby locking the jaws 12a, 12b together in the closed configuration shown in FIG. 10. The extensions provided by the tabs 16a allow a tool, such as endoscopic graspers, or other clamping type tool that can be operated from outside of the body during a minimally invasive procedure, to engage the tabs and clamp or compress them towards one another. This causes the free ends of tabs to also move toward one another, becoming misaligned with the mating features 16b, thereby unlocking the device 10 and allowing jaws 12a, 12b to move apart from one another. This functionality can be useful in many situations, including, but not limited to: unlocking device 10 so as to reposition or reorient it relative to tissues to be clamped, and then re-locking of the device; or removal of the device from its clamped location against tissues in the body. Accordingly, jaws 12a,12b can be locked together as locking features 16a,16b form a locking snap-fit upon compressing the jaws 12a, 12b together, but this locked configuration can be unlocked by compressing the tabs 16a together. Advantageously, locking and unlocking are repeatable. Other mechanisms for automatically locking jaws 12a,12b together upon closing the jaws to relative positions as shown in FIG. 10 may be substituted, as would be readily apparent to one of ordinary skill in the art.

Compressible material 18 may optionally line the inside surfaces of jaws 12a,12b to provide a compliant clamping action against the outside surfaces of the base of an atrial appendage, when device 10 is closed and locked around such an appendage, thereby clamping the walls together and closing off the chamber within the atrial appendage from blood flow to or from the main chamber of the atrium from which the appendage extends. Compliant material 18 may be provided in the way of elastomeric tubing slid over portions 12a,12b, or layers of compressible material 18 may be formed or adhered to the inside surfaces 150, 152 of portions 12a,12b to add compliance to the clamping action. For example, a layer of compressible, open or closed-cell foam (e.g., made from an elastomeric material, such as silicone rubber, polyurethane, C-FLEX™ (silicone-based copolymer), or the like) may be adhered to the inner surface of each jaw 12a,12b. Alternatively, the compressible material 18 may be dovetailed into a slot in jaw 12a,12b to connect it thereto. FIG. 10 shows device 10 in a closed and locked configuration, the configuration that is maintained by device 10 around the base of an atrial appendage upon completion of a ligation procedure.

FIG. 11 shows device 10 in an open configuration, device 10 having been mounted to a delivery tool 40, wherein a distal end portion of delivery tool 40 is shown in FIG. 2. A closure driver 30, such as a suture, flexible wire, cable, or the like is threaded through one of jaws 12a,12b near joint 14 and through an opposite end portion of that jaw, through an open end portion of the opposite jaw and then through the opposite jaw at the end near joint 14 so as to substantially encircle the open jaws longitudinally. One end of closure driver 30 may be fixed to the end portion of the device 10 that the hinge is located at, with the other end extending into tool 40 to connect with an actuator, as described in more detail below. Alternatively, and preferably, both ends of closure driver 30 can extend into tool 40, with one end being connected to the actuator and another fixed relative to tool 40. A portion of closure driver 30 in this case is exposed at least one location along tool 40, so that, after actuating the closure driver to close, and lock device 10 (as described in more detail below) closure driver 30 can be severed at an exposed location along the tool 40, thereby severing the closed loop that had been formed by closure driver 30, and therefore closure driver 30 can be slid out (unthreaded) from device 10 and removed along with the removal of tool 40, as the removal force on tool 40 draws the closure driver 30 along with it.

Device 10 is releasably mounted to the distal end of tool 40 by a tongue and groove type connector 42 that constrains device 10 from moving distally away from or proximally toward the distal end of tool 40 as well as prevents movements perpendicular to these directions. Additionally, to prevent device 10 from rotating with respect to tool 40 about the location of its connection with the tool, a living hinge 44 is releasably fixed against the device, which may be released by application of tension through a tether connected to the living hinge. In FIG. 11, living hinge 44 is shorn engaged against device 10 wherein it presses against flats 14f formed on the outer surface of hinge 14 (e.g., see FIG. 12).

A biasing member 46, such as a leaf spring or the like, is fixed to a distal end portion of tool 40 and extends through an opening in a jaw of the device 10 (jaw 12b in the example shown in FIG. 12) to abut against the opposite jaw (jaw 12a in the example shown in FIG. 12) when device 10 is mounted on tool 40 as described, thereby maintaining the jaws in the open configuration shown in FIG. 11.

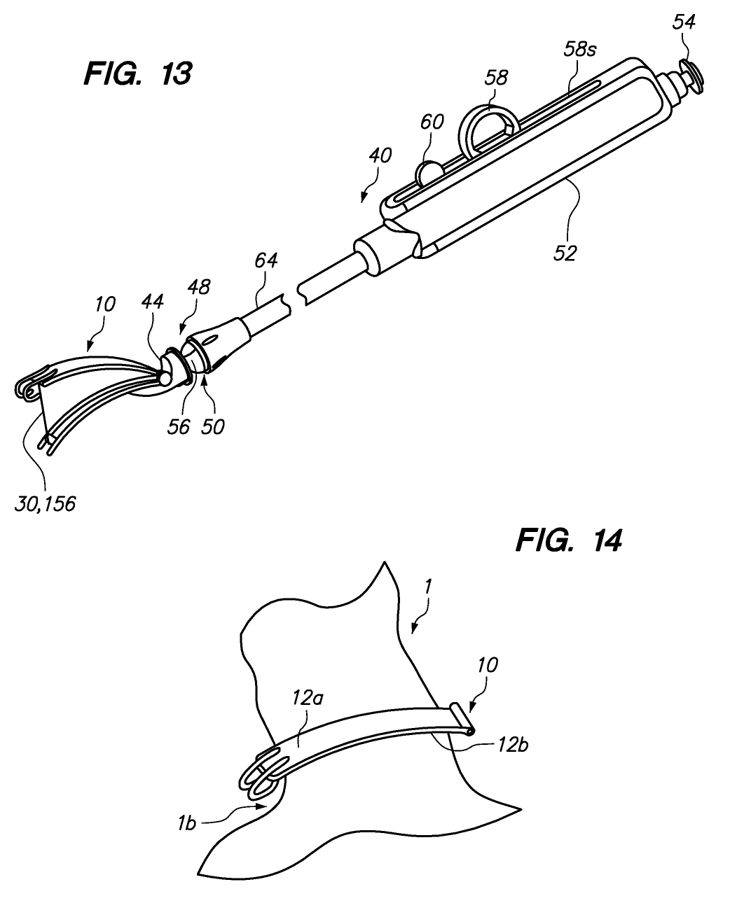

FIG. 13 is a partial view of an assembly having device 10 mounted on tool 40, with an intermediate section omitted, due to the length of the overall tool, including the elongated shaft 64 interconnecting the proximal and distal end portions of tool 40 (required for minimally invasive use through a port to locate the distal end portion at the target surgical site while the proximal end portion extends out of the body of the patient), so that the distal and proximal end portions can be shown in greater detail. The distal platform 48 that device 10 is mounted to is fixed to an articulating joint 50 included in the distal end portion of tool 40. An actuator 54 is provided on handle 52. Actuator 54 is connected to platform 48 via control spires 56 which extend past articulating joint 50 and connect directly to platform 48, so that movement of actuator 54 causes articulation of the platform 48, and consequently reorientation of device 10 relative to the longitudinal axis of device 40. In the example shown, actuator 54 functions like a joystick and connects to platform 48 via four control wires spaced apart angularly by ninety degrees each. In this arrangement, device 10 can be angulated relative to the longitudinal axis of tool 40 up to a maximum of about eighty degrees in any direction.

Further described herein are methods of implanting device 10 in a subject. In certain embodiments, the method comprises: (1) providing an assembly comprising an implantable device 10 having an elongate body movably mated with an elongate shaft 20; (2) inserting the assembly with the elongate body of the implantable device 10 positioned substantially parallel to the elongate shaft 20; (3) moving the body of the implantable device 10 relative to the elongate shaft 20; (4) clamping tissue between clamping members 12a, 12b of the implantable device 10; and (5) detaching device 10 and removing elongate shaft 20.

In certain embodiments, the assembly is inserted via a left or right intercostal incision, or via a subxyphoid incision. The assembly may be inserted alone: through a deliver) cannula or trocar, or in conjunction with a laparoscope, an arthroscope, an endoscope, or any other device useful for monitoring and visualizing the path of the assembly as it is guided through the body cavity towards the LAA. In certain other embodiments, implantable device 10 may further comprise a groove or depression in first 12a or second 12b clamping member, or both. Furthermore, first 12a or second 12b clamping member, or both, may be lined with rubber or any other material commonly used to improve grip or the ability to hold tissue in place after the implantable device 10 is closed and locked.

In yet another embodiment, a tissue barrier can separate tissue positioned between the first and second clamping members from the device locking mechanism. As the first and second clamping members converge to clamp tissue, the tissue barrier can inhibit pinching of tissue in the device locking mechanism. In one aspect, the tissue barrier is formed of resilient material and stretches as the first and second members diverge from one another, and returns to its original configuration as the clamping members converge. In another aspect, the tissue barrier is formed of rigid or semi-rigid material and passes through an aperture in one of the clamping member as the clamping members converge.

After the surgical field is made ready and a sterile delivery cannula is provided, a user can load a sterile tool 40 with device 10 in the insertion configuration. Next, a subxyphoid incision of appropriate size to permit introduction of the delivery cannula and an endoscope is made under the patient's stemum. The delivery cannula, including the LAA occluding device delivery tool 40 loaded with an implantable device 10, is introduced through the incision accompanied by an endoscope. Using the images provided by the endoscope and the illumination provided by the delivery cannula, a surgeon carefully guides the LAA occluding device delivery tool 40 and device 10 to the heart. After the LAA is located, the surgeon translates a handle of the tool 40 and moves the implantable device 10 from the substantially co-axial or co-linear insertion configuration to the implantation/clamping configuration. The surgeon then unlocks and opens the implantable device 10, places it at the base of the LAA, inserts the LAA between the first and second members 12a, 12b, and closes and locks device 10. The opening/closing and locking/unlocking of device 10 can be achieved, for example, by manipulating rod 28 (e.g., moving the rod in a proximal/distal direction and/or rotating rod 28). Next, the surgeon cuts and removes the filament attaching the implantable device 10 to rod 28 and thereby detaches rod 28 from device 10. The surgeon can also detach the locked device from shaft 20. In one aspect, the surgeon pushes a button (or other control mechanism) to rotate latch 146 and thereby release the implantable device 10 from the elongate shaft 20. Shaft 20 and rod 28 are then removed and the subxyphoid incision is closed.

Placement of the implantable device 10 and occlusion of the LAA can be monitored periodically after the surgery to ensure the patient remains free of post-surgical complications, and to assess the efficacy of LAA occlusion in reducing the risk of stroke compared to a similarly situated patient with a normal (i.e., unoccluded) LAA.

In the configuration shown in FIG. 13, device 10 can be inserted through a small opening, such as a port or cannula, or small incision, to deliver the device to a target surgical area where tissues are to be clamped together, maneuver the device 10 over the tissues to be clamped, and close and lock device 10 around the tissues to be clamped, thereby clamping the tissues together to prevent blood or other fluid flow between the clamped tissues. After clamping has been performed to the satisfaction of the surgeon, closure driver 30 can be severed and device 10 can be disengaged from tool 40 (described in more detail below), after which tool 40 can be withdrawn from the patient, to complete the ligation procedure.

If the open configuration of device 10 is too large to fit through a small port, then the open ends of the jaws can be closed down to reduce the dimension thereof sufficiently to allow insertion into and through the port. Upon extending out of the distal end of the port, device 10 returns to the open configuration shown in FIG. 4A, as driven open by the biasing of biasing member 46. Alternatively, tension can be maintained on closure driver 30 to maintain jaws 12a,12b substantially closed (with or without locking) until device 10 has reached or at least traveled closer to the target tissue to be occluded, after exiting the distal end of the port. As noted, device 10 can be reoriented to an appropriate orientation for placement over the target tissues by operation of actuator 54. Once positioned over tissues to be clamped in a desired location (e.g., for atrial appendage ligation, device 10 is slid over the atrial appendage so that jaws 12a, 12b are placed against opposite %% all of the atrial appendage, and device 10 is slid down around the base of the atrial appendage, where it is closed and locked), device 10 can be closed and locked by operation of actuator 53. Actuator 58 has one end of closure driver 30 connected thereto, and, When slid proximally relative to handle 52 in slot 58s, draws closure driver 30 proximally with it, thereby closing the jaws 12a, 12b together, since the other end of closure driver is fixed relative to tool 40 and handle 52. Thus, jaws 12a,12b are driven together until the components of locking mechanism 16 automatically engage each other, in a manner as described above, and automatically lock jaws 12a, 12b into their closed, locked configuration (illustrated in FIG. 10). The closing and locking of jaws 12a,12b together also biases biasing member 46 away from the jaws 12a, 12b, adding potential energy to the biasing member 46.

Device 10 can then be released from tool 40 by severing closure member 30 and releasing the other mechanical connections between tool 40 and device 10. Typically, closure member 30 is severed prior to releasing living hinge 44. To release the other mechanical connections, actuator 60, which is connected to living hinge 44 by tether 62 (see FIG. 12) is slid proximally relative to handle 52. This pulls the living hinge 44 proximally also, via tether 62. Thus, the proximal movement of living hinge 44 causes it to slide off of flats 14f, thereby allowing device 10 to rotate relative to the platform 48. The potential energy stored in biasing member 46 is then converted to kinetic energy: driving the platform 48 and device 10 away from one another and releasing the tongue and groove connection. Closure driver 30 can be removed by severing it and removing it from device 10 and from the patient, by sliding it out of the locations on device that it was threaded through. For embodiments in which the opposite end of closure driver is fixed to tool 40, an exposed portion of closure driver 30 (e.g., exposed in slot 58s distally of actuator 58) can be severed, and then, upon withdrawing tool 40, closure driver 30 is drawn out along with tool 40. FIG. 14 illustrates a device 10 clamped against opposite walls of an atrial appendage 1, near the base 1b of the appendage, by a procedure as described above.

As noted previously, if the initial closure and locking of device 10 around the target tissues does not meet the approval of the surgeon for some reason, then prior to the disconnection from the tool 40 and severing of closure driver 30, device can be re-opened by compressing the tabs 16a together to release the lock. Repositioning and relocking can then be performed, in the manners described, and tool 40 can thereafter be removed.

While the present invention has been described with reference to the specific embodiments thereof, it should be understood by those skilled in the art that various changes may be made and equivalents mat, be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. A device for occluding fluid flow between two walls of tissue in a patient, said device comprising:
a first jaw configured to apply compressive force against a first of the two walls; and
a second jaw configured to apply compressive force against a second of the two walls upon installing the device, wherein, when installed, said first and second jaws compress the two walls therebetween;
said first and second jaws having an open configuration, in which first end portions of said first and second jaws are joined by a joint and second end portions of said first and second jaws are separated, said first and second jaws being movable to a closed configuration in which said first end portions are joined by said joint and said second end portions are connected by a locking mechanism.

2. The device of claim 1, wherein said locking mechanism comprises an automatic locking mechanism.

3. The device of claim 1, wherein the two walls of tissue are opposing walls at the base of an atrial appendage.

4. The device of claim 2, wherein said automatic locking mechanism is actuatable to unlock the device to allow movement of said jaws from said closed configuration to said open configuration.

5. The device of claim 1, further comprising a closure driver mechanically connected to said first and second jaws, said closure driver being actuatable from a location outside of a patient, to move said first and second jaws from said open configuration to said closed configuration when said device is located internally of the patient.

6. The device of claim 5, wherein said closure driver comprise a suture, wire, cable or thread threaded thorough said first and second end portions of said first and second jaws to substantially longitudinally surround said first and second jaws.

7. The device of claim 1, said device further comprising a first and a second mating surface for detachably mating the device to a delivery tool.

8. The device of claim 1, wherein said locking mechanism extends from a face of said first jaw that opposes a face of said second jaw.

9. The device of claim 8, wherein said locking mechanism extends through an aperture in said second jaw when said first and second jaws are locked.

10. The device of claim 9, wherein said locking member rotatably mates with a member on said face of said first jaw that opposes a face of said second jaw.

11. The device of claim 8, wherein the first mating surface is defined by a portion of the locking member.

12. The device of claim 1, further comprising a tissue barrier extending between the first and second clamping members.

13. The device of claim 12, wherein the tissue barrier is positioned between tissue clamping surfaces of said jaws and said locking mechanism.

14. A tool for minimally invasive delivery and installation of an occlusion device, said tool comprising:
an elongated shaft connecting distal and proximal end portions, said tool being configured and dimensioned to deliver said distal end portion through a small opening in a patient, to a reduced-access surgical location, while said proximal end portion of said tool remains outside of the patient;
said distal end portion including a platform configured to releasably engage the occlusion device; and
said proximal end portion includes a release actuator actuatable from outside of the patient, to release the device from said distal end portion located in the reduced-access surgical location inside the patient.

15. The tool of claim 14, wherein said proximal end portion further includes a closure actuator actuatable from outside of the patient, to close portions of the device together around target tissues, thereby clamping them.

16. The tool of claim 14, wherein said proximal end portion further comprises a platform control actuator actuatable from outside of the patient, to articulate said platform relative to said elongated shaft, when said platform is located inside the patient.

17. An assembly comprising:
a device releasably mounted to a distal end portion of a tool, said device and tool configured and dimensioned for delivery of the device through a minimally invasive opening in a patient to a target surgical site, while a proximal end portion of the tool remains outside of the patient, for occluding fluid flow between two walls of tissue in a patient;
said device comprising a first jaw configured to apply compressive force against a first of two walls of tissue to be compressed together and a second jaw configured to apply compressive force against a second of the two walls upon installing the device, said first and second jaws connected at first end portions thereof by a joint;
said tool comprising an elongate shaft connecting distal and proximal end portions, said distal end portion including a mating feature configured to releasably mate with said device.

18. The assembly of claim 17, further comprising an articulating mechanism extending along at least a portion of said shaft for moving said device relative to said shaft.

19. The assembly of claim 17, wherein said mating feature comprises a platform configured to releasably engage said device, and said proximal end portion including a release actuator actuatable from outside of the patient, to release the device from said distal end portion when located in the target surgical site inside the patient, said device being releasably connectable to said platform.

20. The assembly of claim 17, wherein said mating feature allows relative movement of said device with respect to said shaft and detachably connects said device and said shaft.

21. The assembly of claim 19, Wherein said first end portions are releasably mounted to said platform.

22. The assembly of claim 17, wherein second end portions of said jaws are spaced apart from one another, in an open configuration of said device.

23. The assembly of claim 17, further comprising a closure driver linked with said device and actuatable from said proximal end portion of said tool, from a location outside of said patient, to close said jaws when said jaws are located at said target surgical site.

24. The assembly of claim 23, wherein said closure driver comprises a suture, wire, cable or thread having one end connected to a closure actuator at said proximal end portion of said tool, and a second end fixed relative to said tool.

25. The assembly of claim 23, wherein said closure driver has one end connected to a closure actuator at said proximal end portion of said tool, and a second end fixed relative to said tool.

26. The assembly of claim 23, wherein said closure driver substantially surround said first and second jaws in a longitudinal direction.

27. The assembly of claim 19, further comprising an articulating joint connecting said platform with said elongated shaft.

28. The assembly of claim 27, wherein said proximal end portion of said tool further comprises a platform control actuator, said platform control actuator being linked with said platform, whereby actuation of said platform control actuator from a location outside of the body of the patient controls articulation of said platform, via said articulating joint, when said platform is located at said target surgical site within the patient.

29. The assembly of claim 17, further comprising a mechanical linkage between said tool and said device, said mechanical linkage releasably connecting said device to said tool.

30. The assembly of claim 29, wherein said proximal end portion of said tool further comprises a release actuator linked with said mechanical linkage and actuatable to release said mechanical linkage to release said device from said platform.

31. The assembly of claim 17, wherein said device can move between a first position where said shaft and said device are substantially co-linear and a second position where said device is positioned at an angle with respect to said shaft.

32. The assembly of claim 17, further comprising a tissue barrier extending between said jaws.

33. The assembly of claim 17, wherein said elongate shaft comprises a first elongate shaft, said assembly further comprising:
a second elongate shaft, said mating feature comprising a first mating surface on said first elongate shaft for movably mating with said device and a second mating surface on said second elongate shaft;
said device comprising a first device mating surface for detachably mating with said first mating surface of said first shaft, and a second device mating surface for detachably mating with said second mating surface of said second shaft;
wherein movement of said first or second shaft relative to the other of said first and second shaft moves said clip with respect to said tool.

34. The assembly of claim 17, wherein said first shaft extends through an aperture in said second jaw.

35. The assembly of claim 17, wherein movement of said first shaft locks and unlocks said first and second jaws relative to one another.

36. A method of performing an occlusion of fluid flow between two walls of tissue in a patient said method comprising:
inserting an occlusion device connected to a tool through a minimally invasive opening in a patient;
delivering the occlusion device to a location of the two walls to be occluded;
positioning opposite jaws of the device against the two walls, respectively;
clamping the walls between the jaws by closing and locking the jaws; and
releasing the device from the tool.

37. The method of claim 36, further comprising articulating the device relative to a shaft of the tool to position the device in a desired orientation for placement of the jaws against the tissue walls.

38. A method of implanting a clip comprising:
providing an implant system comprising an implantable clip having an elongate body and movably mated with an elongate shaft that extends between a proximal and distal end;
inserting the implantable clip through a surgical opening while the clip is positioned in a low profile configuration;
moving the implantable clip relative to the shaft after insertion to position the clip for clamping;
clamping tissue between clamping members of the implantable clip; and
detaching the clip and removing the elongate shaft.

39. A method of articulating and detaching a medical device comprising:
providing an elongate clamp body extending between a proximal end and a distal end and comprising first and second clamping members having first and second opposing surfaces, the clamping members movably mated with one another proximate to the distal end of the clamp body, the clamping members mated with first and second shafts;
moving the first shaft relative to the second shaft to cause the clamp to move relative to the second shaft; and
moving the first shaft relative to the second shaft to move the first clamping member relative to the second clamping member.
